(19) United States
(12) Patent Application Publication
(10) Pub. No.: US 2022/0288148 A1
(43) Pub. Date: Sep. 15, 2022

(54) METHODS FOR RELIEVING BACK PAIN WITH TERMINALIA CHEBULA COMPOSITIONS

(71) Applicant: Natreon, Inc., New Brunswick, NJ (US)

(72) Inventor: Sanyasi R. Kalidindi, Monroe, NJ (US)

(73) Assignee: Natreon, Inc., New Brunswick, NJ (US)

(21) Appl. No.: 17/690,837

(22) Filed: Mar. 9, 2022

Related U.S. Application Data
(60) Provisional application No. 63/158,412, filed on Mar. 9, 2021.

(51) Int. Cl. A61K 36/61; A61P 29/00

(52) U.S. Cl. CPC A61K 36/61; A61P 29/00

(57) ABSTRACT

Methods of relieving back pain with Terminalia chebula compositions are described.

### CROSS-REFERENCE TO RELATED APPLICATIONS

This application claims the benefit of U.S. Provisional Application No. 63/158,412 filed on Mar. 9, 2021 which is incorporated by reference herein in its entirety.

### FIELD OF THE INVENTION

This invention relates to methods for relieving back pain with Terminalia chebula compositions.

### BACKGROUND

Back pain, also known as backache, is pain felt in the back. Back pain may be divided into neck pain (cervical), middle back pain (thoracic), lower back pain (lumbar) or co4cydynia (tailbone or sacral pain). Back pain may be acute, sub-acute, or chronic, depending on the duration. The pain may be characterized as a dull ache, a shooting or piercing pain, or a burning sensation. Discomfort can radiate into the arms and hands as well as the legs or feet, and may include numbness or weakness in the legs and arms.

The majority of back pain is nonspecific with no identifiable causes. Common underlying mechanisms include degenerative or traumatic changes to the disks and facets joints, which can then cause secondary pain in the muscles, and nerves, and referred pain to the bones, joints and extremities. Diseases and inflammation of the gallbladder, pancreas, aorta, and kidneys may also cause referred pain in the back. Tumors of the vertebrae, neural tissues and adjacent structures can also manifest as back pain.

Back pain is common, with about nine out of ten adults experiencing it at some point in their life, and five out of ten working adults having it every year. Some estimate up to 95% of people will experience back pain at some point in their lifetime. It is the most common cause of chronic pain, and is a major contributor of missed work and disability.

For most individuals, back pain is self-limiting. In most cases of herniated disks and stenosis, rest, injections, or surgery have similar general pain resolution outcomes on average after one year. In the United States, acute low back pain is the fifth most common reason for physician visits and causes 40% of missed days off work. Additionally, it is the single leading cause of disability worldwide.

Back pain may be classified in terms of duration of symptoms.

Acute back pain lasts less than 6 weeks.

Subacute back pain lasts 6 weeks to 12 weeks.

Chronic back pain lasts for more than 12 weeks.

There are many causes of back pain, including blood vessels, internal organs, infections, mechanical, and autoimmune causes. Approximately 90 percent of people with back pain are diagnosed with nonspecific acute back pain in which there is no identifiable underlying pathology. In approximately 10 percent of people, a cause can be identified through diagnostic imaging. Less than 2 percent are attributed to secondary factors, with metastatic cancers and serious infections, such as spinal osteomyelitis and epidural abscesses, accounting for around 1 percent.

### Nonspecific Pain:

In as many as 90 percent of cases, no physiological causes or abnormalities on diagnostic tests can be found. Nonspecific back pain can be due to back strain/sprain. The cause is peripheral injury to muscle or ligaments. The patient may or may not recall the cause. The pain can present acutely, but in some cases can persist, leading to chronic pain. Chronic back pain in people with otherwise normal scans can result from central sensitization, where an initial injury causes a longer-lasting state of heightened sensitivity to pain. This persistent state maintains pain even after the initial injury has healed. Treatment of sensitization may involve low doses of anti-depressants and directed rehabilitation such as physical therapy.

### Spinal Disk Disease:

Spinal disk disease occurs when the nucleus pulposus, a gel-like material in the inner core of the vertebral disk, ruptures. Rupturing of the nucleus pulposus can lead to compression of nerve roots. Symptoms may be unilateral or bilateral, and correlate to the region of the spine affected. The most common region for spinal disk disease is at L4-L5 or L5-S1. The risk for lumbar disk disease is increased in overweight individuals due to the increased compressive force on the nucleus pulposus.

### Lumbar Disk Herniation:

Severe spinal cord compression is considered a surgical emergency and requires decompression to preserve motor and sensory function. Cauda equina syndrome refers to severe compression of the cauda equina and presents initially with pain followed by motor weakness and sensory dysfunction.

### Degenerative Disease:

Spondylosis, or degenerative arthritis of the spine, occurs when the intervertebral disk undergoes degenerative changes, causing the disk to fail at cushioning the vertebrae. There is an association between intervertebral disk space narrowing and lumbar spine pain. The space between the vertebrae becomes more narrow, resulting in compression and irritation of the nerves.

Spondylolisthesis is the anterior shift of one vertebra compared to the neighboring vertebra. It is associated with age-related degenerative changes as well as trauma and congenital anomalies.

Spinal stenosis can occur in cases of severe spondylosis, spondylolisthesis, and age associated thickening of the ligamentum flavum. Spinal stenosis involves narrowing of the spinal canal and typically presents in patients greater than 60 years of age. Neurogenic claudication can occur in cases of severe lumbar spinal stenosis and presents with symptoms of pain in the lower back, buttock or leg that is worsened by standing and relieved by sitting.

Vertebral compression fractures occur in 4% of patients presenting to primary care with low back pain. Risk factors include age, female gender, history of osteoporosis, and chronic glucocorticoid use. Fractures can occur due to trauma but in many cases can be asymptomatic.

### Infection:

Common infectious causes of back pain include osteomyelitis, septic diskitis, paraspinal abscess, and epidural abscess. Infectious causes that lead to back pain involve various structures surrounding the spine.

Osteomyelitis is the bacterial infection of the bone. Vertebral osteomyelitis is most commonly caused by staphylococci. Risk factors include skin infection, urinary tract infection, IV catheter use, IV drug use, previous endocarditis, and lung disease.

Spinal epidural abscess is also commonly caused by severe infection with bacteremia. Risk factors include recent epidural, IV drug use, or recent infection.

### Cancer:

The spread of cancer to the bone or spinal cord can lead to back pain. Bone is one of the most common sites of metastatic lesions. Patients typically have a history of malignancy. Common types of cancer that present with back pain include multiple myeloma, lymphoma, leukemia, spinal cord tumors, primary vertebral tumors, and prostate cancer. Back pain is present in 29% of patients with systemic cancer. Unlike other causes of back pain which commonly affect the lumbar spine, the thoracic spine is most commonly affected. The pain can be associated with systemic symptoms such as weight loss, chills, fever, nausea and vomiting. Unlike other causes of back pain, neoplasm-associated back pain is constant, dull, poorly localized, and worst with rest. Metastasis to the bone also increases the risk of spinal cord compression or vertebral fractures which requires emergent surgical treatment.

### Autoimmune:

Back pain can be caused by the vertebrae compressing the intervertebral disks.

Inflammatory arthritides, such as ankylosing spondylitis, psoriatic arthritis, rheumatoid arthritis, and systemic lupus erythematosus, can all cause varying levels of joint destruction. Among the inflammatory arthritides, ankylosing spondylitis is most closely associated with back pain due to the inflammatory destruction of the bony components of the spine. Ankylosing spondylitis is common in young men and presents with a range of possible symptoms such as uveitis, psoriasis, and inflammatory bowel disease.

### Referred Pain:

Back pain can also be due to referred pain from another source. Referred pain occurs when pain is felt at a location different from the source of the pain. Disease processes that can present with back pain include pancreatitis, kidney stones, severe urinary tract infections, and abdominal aortic aneurysms.

### Risk Factors:

Heavy lifting, obesity, sedentary lifestyle, and lack of exercise can increase a person's risk of back pain. People who smoke are more likely to experience back pain than others. Poor posture and weight gain in pregnancy are also risk factors for back pain. In general, fatigue can worsen pain.

A few studies suggest that psychosocial factors such as on-the-job stress and dysfunctional family relationships may correlate more closely with back pain than structural abnormalities revealed in X-rays and other medical imaging scans.

### Management of Pain:

### Nonspecific Pain

Patients with uncomplicated back pain may be encouraged to remain active and return to normal activities.

Management goals when treating back pain are to achieve maximal reduction in pain intensity as rapidly as possible, to restore the individual's ability to function in everyday activities, to help the patient cope with residual pain, to assess for side-effects of therapy, and to facilitate the patient's passage through legal and socioeconomic impediments to recovery. For many, the goal is to keep pain to a manageable level to progress with rehabilitation, which then can lead to long-term pain relief. Also, for some people the goal is to use non-surgical therapies to manage pain and avoid major surgery, while for others surgery may be the quickest way to feel better.

Not all treatments work for all conditions or for all individuals with the same condition, and many find that they need to try several treatment options to determine what works best for them. The present stage of the condition (e.g. acute or chronic) is also a determining factor in the choice of treatment. Only a minority of people with back pain (most estimates are 1%-10%) require surgery.

### Non-Medical

Back pain is generally treated with non-pharmacological therapy first, as it typically resolves without the use of medication. Superficial heat and massage, acupuncture, and spinal manipulation therapy may be recommended.

Heat therapy is useful for back spasms or other conditions. A review concluded that heat therapy can reduce symptoms of acute and sub-acute low-back pain.

Regular activity and gentle stretching exercises are encouraged in uncomplicated back pain, and are associated with better long-term outcomes. Physical therapy to strengthen the muscles in the abdomen and around the spine may also be recommended. These exercises are associated with better patient satisfaction, although it has not been shown to provide functional improvement. Exercise may be effective for chronic back pain, but not for acute pain. If used, these exercises should be performed under supervision of a licensed health professional.

Massage therapy may give short-term pain relief, but typically not functional improvement, for those with acute lower back pain. It may also give short-term pain relief and functional improvement for those with long-term (chronic) and sub-acute lower back pain, but this benefit does not appear to be sustained after 6 months of treatment.

Acupuncture may provide some relief for back pain. However, further research with stronger evidence needs to be done.

Spinal manipulation appears similar to other recommended treatments.

“Back School” is an intervention that consists of both education and physical exercises. There is no strong evidence supporting the use of Back School for treating acute, sub-acute, or chronic non-specific back pain.

Insoles appear to be an ineffective treatment intervention.

While traction for back pain is often used in combination with other approaches there appears to be little or no impact on pain intensity, functional status, global improvement, and return to work.

### Medications

If non-pharmacological measures are not effective, medications may be tried.

Non-steroidal anti-inflammatory drugs (NSAIDs) are typically tried first. NSAIDs have been shown to be more effective than placebo and are usually more effective than acetaminophen.

Long-term use of opioids has not been tested for treating chronic lower back pain. For severe back pain not relieved by NSAIDs or acetaminophen, opioids may be used. Opioids may not be better than NSAIDs or antidepressants for chronic back pain with regards to pain relief and gain of function. In addition, there is a possibility of increased tolerance and addiction potential with opioids.

Skeletal muscle relaxers may also be used. Their short term use has been shown to be effective in the relief of acute back pain. However, the evidence of this effect has been disputed, and these medications may have negative side-effects.

In people with nerve root pain and acute radiculopathy, there is evidence that a single dose of steroids, such as dexamethasone, may provide pain relief.

Epidural corticosteroid injection (ESI) is a procedure in which steroid medications are injected into the epidural space. The steroid medications reduce inflammation and thus decrease pain and improve function. Steroid injections contain various formulations of medications. A common combination is a numbing drug, similar to procaine (Novocain), mixed with the anti-inflammatory drug cortisone. Corticosteroid injections do not change the course of a chronic back pain condition. ESI has long been used to both diagnose and treat back pain, although recent studies have shown a lack of efficacy in treating low back pain.

There is an urgent need for a product, preferably without any serious adverse effects, for treatment of low back pain.

Terminalia chebula (T. chebula, chebulic/black myrobalan) is an Ayurvedic medicinal plant and its fruit powder is a constituent in many Ayurvedic formulations. Terminalia chebula (T. chebula) is rich in low molecular weight hydrolysable tannoids or tannins (LMwHTs). The main tannoids/tannins in T. chebula fruit are chebulinic acid, chebulagic acid, corilagin and a tannoid metabolite, gallic acid. Other minor hydrolyzable tannoids reported in T. chebula include punicalagin, chebulanin, neochebulinic acid, 1,2,3,4,6-penta-O-galloyl-β-D-glucose, 1,6-di-O-galloyl-D-glucose, casuarinin, 3,4,6-tri-O-galloyl-D-glucose, and terchebulin. T. chebula may have a tannoid content of about 32% by weight. Other constituents of T. chebula may include fructose, amino acids, succinic acid, beta-sitosterol, resin and purgative principles of anthroquinone, sennoside, flavonol glycosides, triterpenoids and coumarin conjugated with gallic acids.

T. chebula fruits contain a variety of hydrolysable tannins (for instance gallic acid, chebulic acid, punicalagin, chebulanin, corilagin, neochebulinic acid, ellagic acid, chebulagic acid, chebulinic acid, 1,2,3,4,6-penta-O-galloyl-b-D-glucose, casuarinin, 3,4,6-tri-O-galloyl-D-glucose and terchebulin).

T. chebula fruit extract has been shown to have antioxidant, anthelminthic, antibacterial, antiviral, antifungal, anticancer, antinociceptive, antiarthritic, antiulcerogenic, cytoprotective, and radioprotective activities.

The effect of a standardized ethanolic extract of T. chebula, NDI10218, was studied on collagen-induced arthritis in an acetic acid-induced writhing model. NDI10218 reduced the arthritis index and blocked synovial hyperplasia in a dose-dependent manner. Serum levels of pro-inflammatory cytokines TNF-α, IL-6, and IL-1β were significantly reduced in mice treated with NDI10218. Production of the inflammatory IL-17, but not immunosuppressive IL-10, was also inhibited in splenocytes isolated from NDI10218-treated arthritic mice. Administration of NDI10218 markedly decreased the number of T cell sub populations in the regional lymph nodes of the arthritic mice. Finally, NDI10218 reduced the number of abdominal contractions in acetic acid-induced writhing model, suggesting an analgesic effect of this extract. Taken together, these results suggest that NDI10218 can be a new therapeutic candidate for the treatment of rheumatoid arthritis. SEO Jong Bae, et al., “Anti-Arthritic and Analgesic Effect of NDI10218, a Standardized Extract of Terminalia chebula, on Arthritis and Pain Model” Biomol. Ther. 20(1):104-112 (2012).

Chebulagic acid and chebulinic acid are main LMwHTs in T. chebula. Chebulagic acid is a natural antioxidant and has shown anti-inflammatory effects in a mouse macrophage cell line. D. B. Reddy, et al., Biochemical and Biophysical Research Communications. 381:112-117 (2009).

The effect of Chebulinic acid was studied on mice with collagen-induced arthritis. L U et al., Arthritis Res Ther. 22: 273 (2020). The conclusion was that chebulinic acid can act as a safe and potent anti-VEGF antiangiogenic agent for the treatment of types of inflammatory arthritis, such as rheumatoid arthritis.

A hydro-alcoholic extract of T. chebula was proposed to have a potent anti-arthritic activity based on a study in rats. NAIR et al., J Pharm Pharmacol. 2010; 62(12):1801-1806.

### Clinical Studies

The composition of the present invention (AyuFlex®) has been the subject of several human clinical studies as well as in vitro and in vivo studies.

A hyperuricemia study was conducted with T. chebula fruit extract and Terminalia bellerica (T. bellerica) fruit extract versus a placebo. USHARANI PINGALI et al., Clinical Pharmacology 8:51-9 (2016). A total of 110 eligible subjects with hyperuricemia were enrolled and randomized to either of the five treatment groups—T. chebula 500 mg twice a day (BID), T. bellerica 250 mg BID, T. bellerica 500 mg BID, placebo BID, and febuxostat 40 mg once daily plus an identical placebo—for a duration of 24 weeks. Serum uric acid levels were measured at baseline and at the end of 4, 8, 12, 16, 20, and 24 weeks. All active treatment groups showed a reduction in serum uric acid levels compared to baseline and placebo. Significant reduction in mean serum uric acid levels started as early as 4 weeks following treatment, compared to baseline, with T. bellerica (500 and 250 mg), febuxostat (P,0.001), and T. chebula 500 mg (P,0.01); an increase in serum uric acid levels was seen with placebo (P,0.05). The serum uric acid levels became steady after 16 weeks of treatment and remained the same until the end of 24 weeks. The reduction of serum uric acid levels in the T. bellerica 500 mg group was nearly twice that of the T. chebula 500 mg group as well as T. bellerica 250 mg group at all time points. T. bellerica 500 mg reduced serum uric acid levels from 8.07±0.87 to 5.78±0.25 compared to febuxostat, which reduced serum uric acid levels from 8.53±0.97 to 4.28±0.67 (P,0.001) at the end of 24 weeks. All the formulations were well tolerated.

Metabolic syndrome subjects have endothelial dysfunction via increased oxidative stress increasing the risk of atherosclerosis and coronary heart disease. KISHORE et al., EJBPS, Volume 3, Issue 2, 181-188 (2016). T. chebula is known for its antioxidant and antihyperlipidemic activity. A randomized, double-blind, placebo-controlled study compared the effects of an aqueous extract of T. chebula 250 mg and 500 mg versus placebo on endothelial dysfunction and biomarkers of oxidative stress in patients with metabolic syndrome. Eligible patients were randomized to receive either T. chebula 250 mg, T. chebula 500 mg, or placebo twice daily for 12 weeks. The primary efficacy parameter was the change in endothelial function at baseline and after 12 weeks of treatment. Secondary efficacy parameters were changes in biomarkers of oxidative stress (malondialdehyde, nitric oxide, and glutathione), high sensitivity C-reactive protein levels and lipid profile. Laboratory safety parameters were measured at baseline and after 12 weeks of treatment. 56 patients completed the study. Treatment with T. chebula 250 mg and 500 mg for 12 weeks produced significant reductions in the reflection index (−2.25%±0.70% to −3.72%±1.35% versus −2.35%±0.85% to −6.14%±1.01% respectively), suggesting improvement in endothelial function compared with baseline. There was a significant improvement in biomarkers of oxidative stress and systemic inflammation compared with baseline and placebo. Further, the treatments significantly improved the lipid profile compared with baseline and placebo. All the treatments were well tolerated.

A randomized, double blind, placebo-controlled clinical study was undertaken to evaluate the effects of an aqueous extract of T. chebula 250 mg and 500 mg twice daily versus placebo on endothelial dysfunction and biomarkers of oxidative stress in type 2 diabetic patients. PINGALI et al., Phytotherapy Research. 1-10 (2020). A total of 60 eligible patients were randomized to receive either T. chebula 250 mg, T. chebula 500 mg, or placebo twice daily for 12 weeks. The subjects were assessed based on endothelial function, levels of nitric oxide, malondialdehyde, glutathione, high sensitivity C-reactive protein, glycosylated hemoglobin, and lipid profile at baseline and after 12 weeks of treatment. Treatment with T. chebula 250 mg and T. chebula 500 mg twice daily for 12 weeks significantly improved endothelial function (reflection index) compared to placebo (absolute changes: —T. chebula 250: −2.55±1.82% vs. T. chebula 500: −5.21±2.41% vs. placebo: 1.40±2.11%). Other cardiovascular risk indicators were also significantly ameliorated in treatment groups compared to placebo. In conclusion, T. chebula (especially, 500 mg BID dose) significantly minimized cardiovascular risk factors in patients with type 2 diabetes compared to placebo.

The objective of another randomized, double-blind, placebo-controlled clinical study was to evaluate the analgesic effect of an aqueous extract of T. chebula (TCE), a proprietary chromium complex (PCC), and their combination in subjects with knee joint discomfort. CHANDRASEKHAR NUTALAPATIU et al., Asian J Pharm Clin Res, Vol 9, Issue 3, pages 264-269 (2016). A total of 100 patients with knee joint discomfort were randomized into five treatment groups—TCE 500 mg twice daily (BID), TCE 500 mg BID+PCC 400 μg once daily (OD), PCC 400 μg OD alone, placebo, and TCE 250 mg BID, for 12 weeks. Assessment of symptoms of knee joint pain and discomfort was done by modified Western Ontario and McMaster Universities Arthritis Index (mWOMAC) and knee swelling index (KSI); visual analog scale (VAS) was used for subjective assessment of pain, stiffness, and disability. Absolute reduction in mWOMAC score in TCE 500 mg (19.82±8.35), TCE 500 mg+PCC 400 μg (13.10±5.69), PCC 400 μg (8.30±3.81), placebo (2.45±3.07), and TCE 250 mg (10.47±4.43), respectively, at the end of 12 weeks as compared to the baseline values. Absolute reduction in KSI in TCE 500 mg (28.95±16.82), TCE 500 mg+PCC 400 μg (19.14±9.50), PCC 400 μg (12.7±4.86), placebo (10.03±3.8), and TCE 250 mg (18.24±6.86), respectively, at the end of 12 weeks as compared to the baseline values (p<0.001). Similar results were seen with VAS assessments for pain, stiffness and disability. All the treatments were well tolerated.

The aim of another study was to evaluate the effects of a standardized aqueous extract of T. chebula fruit (AyuFlex®). LOPEZ et al., BMC Complementary and Alternative Medicine 17.475 (2017). One-hundred and five (105) overweight, apparently healthy male and female subjects (35-70 years of age) were pre-screened and randomized to one of three groups for 84 days: placebo, AyuFlex1 (250 mg twice daily) or AyuFlex2 (500 mg twice daily) in a randomized, double-blind, placebo-controlled design. A two-week placebo lead-in period was used to improve data quality/validity. All subjects had no knee joint discomfort at rest, but experienced knee joint discomfort only with activity/exercise of at least 30 on 100 mm Visual Analog Scale (VAS). Primary outcome measures included symptoms of joint health and function as measured by modified-Knee Injury & Osteoarthritis Outcomes Score (mKOOS) global & modified-Western Ontario and McMaster Universities Arthritis Index (mWOMAC) subscales (discomfort, stiffness and function). Secondary outcomes included VAS questionnaires on overall/whole-body joint health, low back health, knee mobility, willingness and ability to exercise, 6-min walk test for distance and range of motion (ROM) of pain-free knee flexion/extension. Tertiary outcome measures included inflammatory (high sensitivity C-reactive protein (hsCRP), tumor necrosis factor (TNF)-α) and extracellular matrix (ECM)/Connective Tissue (COMP) biomarkers, and safety (vital signs and blood markers) & tolerability (Adverse Event (AE)/side effect profiles). Compared to placebo, at day 84 AyuFlex® treatment significantly: 1) improved mKOOS global scores in AyuFlex1+AyuFlex2 (P=0.023), and improved total and physical function subscale of mWOMAC relative to baseline, 2) improved VAS scores for Knee Discomfort with activity/exercise in AyuFlex1+AyuFlex2 (P=0.001) relative to baseline, 3) improved VAS scores for whole-body joint function in AyuFlex1+AyuFlex2 (P<0.029) relative to baseline, 4) improved VAS score for decreased knee joint soreness following leg extension challenge for AyuFlex1 (P=0.022) and AyuFlex2 (P=0.043) relative to baseline, 5) improved 6-min walk performance distance covered (P=0.047) and VAS discomfort (P=0.026) post-6 min walk in AyuFlex1+AyuFlex2 relative to baseline, 6) and tended to decrease COMP levels in AyuFlex1+AyuFLex2 (P=0.104) relative to baseline. All biomarkers of safety remained within normative limits during the study. Low back health tended to improve in the AyuFlex1 and AyuFlex2 group, but failed to reach significance relative to placebo group.

The aim of another study was to evaluate analgesic activity and safety of single oral dose of T. chebula fruit aqueous extract using hot air pain model in healthy human participants. Ciranjeevi Uday Kumar et al., Journal of Clinical and Diagnostic Research. Vol-9(5): FC01-FC04 (2015 May). Randomized, Double blind, Placebo controlled, cross over design was used. 12 healthy human participants were randomized to receive either single oral dose of two capsules of Terminalia chebula 500 mg each or identical placebo capsules in a double blinded manner. Thermal pain was assessed using hot air analgesiometer, to deliver thermal pain stimulus. Mean pain threshold time and mean pain tolerance time were measured (in seconds) at baseline and 180 minutes post product administration. A washout period of two weeks was given for cross-over between the two treatments. Terminalia chebula significantly increased mean pain threshold and tolerance time compared to the baseline and placebo. Mean pain threshold time increased from 34.06±2.63 seconds to 41.00±2.99 seconds s (p<0.001) and mean pain tolerance time increased from 49.67±3.72 seconds to 57.30±3.07 seconds (p<0.001). The increase in mean percentage change for pain threshold time is 20.42% (p<0.001) and for pain tolerance time is 17.50% (p<0.001).

In yet another study, analgesic activity and safety of single oral dose (1000 mg) of Terminalia chebula was evaluated using a mechanical pain model in healthy human volunteers. Venkata Kishan Pokur et al., Journal of Anaesthesiology Clinical Pharmacology, Vol 32, Issue 3 (July-September 2016). Twelve healthy volunteers were randomized to receive either single oral dose of 2 capsules of T. chebula 500 mg each or identical placebo capsules in a double-blinded manner. Mechanical pain was assessed using Ugo Basile Analgesy-meter (Randall-Selitto test) before and 3 hours after administration of the test products. The parameters evaluated were mean pain threshold force and time; mean pain tolerance force and time. A washout period of 1-week was given for crossover between the active and placebo treatments. T. chebula significantly increased the mean percentage change for pain threshold force and time, and pain tolerance force and time compared to placebo (P<0.001). The mean percentage change for pain threshold force and time (20.8% and 21.0%) was increased more than that of pain tolerance force and time (13.4% and 13.4%). No adverse drug reaction was reported with either of the study medications during the study period.

Anti-arthritic and anti-inflammatory efficacy of T. chebula aqueous extract (TCE) was also evaluated in moderately osteoarthritic (OA) companion dogs. MURDOCK et al., J. Veterinar. Sci. Technol. 7(1):1000290 (2016). Dogs with OA received either 500 mg placebo or 500 mg TCE twice daily for 150 days. On a monthly basis, dogs were given a full physical exam and were evaluated for arthritic pain (overall pain, pain upon limb manipulation, and pain after physical exertion), inflammation (erythrocyte sedimentation rate, ESR), and analysis of complete blood count (CBC) and serum biomarkers of liver (bilirubin, ALT, and AST), kidney (BUN and creatinine), and heart and skeletal muscle (CK) functions. Elbow and stifle joints were radiographed on day 0 and day 150 for evaluation of arthritic progression. Dogs given TCE showed significant (P<0.01) reductions in overall pain, pain upon limb manipulation, and pain after physical exertion by day 90, with maximum effects on day 150 (81.2%, 81.5%, and 84.2%, respectively). A marked reduction in ESR coincided with pain reduction in TCE-treated dogs, which was indicative of anti-inflammatory effect of TCE. Radiographic evidence also indicated slowed progression of OA in joints examined. No significant change occurred in physical parameters, CBC parameters, or serum biomarkers in dogs on placebo or treatment, which suggested that TCE was well tolerated. It can be concluded that TCE, by having many active principles (chebulagic acid, chebulinic acid, corilagin, hydrolysable tannoids, etc.) might have provided antioxidant, anti-inflammatory, and anti-arthritic effects in dogs without causing any side effects.

In a recent publication, the mechanism of action of antiarthritic activity of an aqueous extract of the fruits of T. chebula (AyuFlex®) was assessed. KIM Hae Lim, et al., Appl. Sci. 10:8698 (2020). In vitro, AyuFlex® decreased oxidative stress and induction of pro-inflammatory cytokines and mediators as well as matrix metalloproteinases (MMPs), while also increasing the levels of collagen synthesis-related proteins. Mechanistically, AyuFlex® disrupted nuclear factor kappa B (NF-κB) and mitogen-activated protein kinase (MAPK) activation via the inhibition of NF-κB p65 and extracellular regulated protein kinase (ERK) phosphorylation. Ameliorating effects of AyuFlex® were also observed in vivo. AyuFlex® significantly inhibited the MIA-incurred increase in OA symptoms such as oxidative stress, cartilage damage, and changes in cytokines and MMPs revelation in arthrodial cartilage. The results suggested that AyuFlex® attenuates OA progression in vivo.

There have been no studies reported on T. chebula to evaluate its effect on back pain, either acute or chronic.

### BRIEF DESCRIPTION OF THE DRAWINGS

FIG. 1 is a copy of an HPLC chromatogram of a standardized aqueous extract of Terminalia chebula fruit, AyuFlex®, showing peaks or ranges of peaks for Chebulagic Acid (t_{R} 10.00 min), Chebulinic Acid (t_{R} 11.13 min), Other LMwHTs (t_{R} 5-9 min), Gallic Acid (t_{R} 5.01 min), Ellagic Acid (t_{R} 11.43 min).

FIG. 2 is a copy of an HPLC-PDA spectra of Gallic Acid (t_{R} 5.01 min), LMwHT (t_{R} 8.42 min), Chebulagic Acid (t_{R} 10.00 min), Chebulinic Acid (t_{R} 11.13 min), and Ellagic Acid (t_{R} 11.43 min).

### SUMMARY OF THE INVENTION

The present invention is directed to methods for using a natural product which has shown unexpected efficacy in relieving back pain. This product is a composition comprising, consisting essentially of, or consisting of the fruits of an Ayurvedic medicinal plant, Terminalia chebula, also known as black or chebulic myrobalan. The treatment of back pain and relief of back pain by administration of a T. chebula composition according to the present invention provided significant relief from back pain by significant reduction or elimination of back pain in subjects after administration of the T. chebula composition.

In an embodiment, the present invention is directed to methods of providing relief from back pain, in an embodiment low back pain, in a subject by administering to the subject a Terminalia chebula composition according to this invention.

In an embodiment, the present invention is directed to a method of providing relief from chronic low back pain by administering to a subject a Terminalia chebula composition according to this invention. In an embodiment, the present invention is directed to a method of providing relief from acute low back pain by administering to a subject a Terminalia chebula composition according to this invention. In an embodiment, the present invention is directed to a method of providing relief from subacute low back pain by administering to a subject a Terminalia chebula composition according to this invention.

In an embodiment, the present invention is directed to a method of providing relief from sciatic low back pain in a subject by administering to the subject a Terminalia chebula composition according to this invention.

In an embodiment, the present invention is directed to a method of providing relief from referred low back pain in a subject by administering to the subject a Terminalia chebula composition according to this invention.

In an embodiment, the present invention is directed to a method of providing relief from non-specific low back pain in a subject by administering to the subject a Terminalia chebula composition according to this invention.

In an embodiment, the present invention is directed to a method of providing relief from low back pain in a subject by administering to the subject an effective dose of a Terminalia chebula composition comprising an aqueous extract of Terminalia chebula fruits of this invention, for instance in an embodiment, AyuFlex®. In an embodiment, the aqueous extract is described and in an embodiment claimed in U.S. Pat. No. 10,500,240 and/or Canadian Patent No. 2,876,719.

In an embodiment, the present invention is directed to a method of providing relief from low back pain by administering a Terminalia chebula composition according to this invention comprising an alcoholic or hydro-alcoholic extract of Terminalia chebula fruits.

In an embodiment, the present invention is directed to a method of providing relief from non-specific low back pain by administering a Terminalia chebula composition according to this invention comprising dried Terminalia chebula fruit powder.

In an embodiment, the present invention is directed to a method of providing relief from non-specific low back pain by administering a Terminalia chebula composition according to this invention comprising dried and standardized Terminalia chebula fruit powder. In an embodiment, a method of this invention comprises the steps of providing a T. chebula composition of this invention to a subject, and then, administering the composition to the subject to relieve back pain.

### DETAILED DESCRIPTION OF THE INVENTION

The present methods are directed to relieving low back pain in a subject by administering a Terminalia chebula composition according to this invention. The administration of AyuFlex® (available from Natreon, Inc., New Brunswick, N.J.) in subjects resulted in a reduction in low back pain in each of Examples 2-5 below.

The below definitions and discussion are intended to guide understanding but are not intended to be limiting with regard to other disclosures in this application.

References to percentage (%) in compositions of the present invention refers to the % by weight of a given component to the total weight of the composition being discussed, also signified by “w/w”, unless stated otherwise.

“Back pain” according to this invention refers to pain felt in the back. Back pain may be divided into neck pain (cervical), middle back pain (thoracic), lower back pain (lumbar) including coccydynia (tailbone or sacral pain). “Low back pain” according to this invention refers to pain in the lumbar area of a subject's spine or below (including tailbone/sacrum/coccyx as appropriate). In an embodiment, back pain may be acute (lasting less than 6 weeks), subacute (lasting 6-12 weeks), or chronic (lasting more than 12 weeks, including for instance 13 weeks to 50 years, such as 4 months to 5 years, 6 months to 4 years, 9 months to 4 years, 1-5 years: 4, 5, 6, 7, 8, 9, 10, 11, or 12 months to 5, 10, 15, 20, 25, 30, 35, 40, 45, 50 years, and so forth). In an embodiment, back pain may be sciatic, referred, and/or non-specific. In an embodiment, back pain relieved by this invention is non-specific low back pain. In an embodiment, low back pain relieved or treated by this invention is severe, including severe non-specific back pain, and back pain not caused by obesity or problems associated with a subject being overweight.

Tissues related to low back pain are discussed throughout this application, and may include for instance vertebrae, disks, facets joints and other joints, blood vessels such as the aorta, muscles, nerves, bones, extremities, internal organs such as gallbladder, pancreas. In an embodiment, methods of the present invention are directed to relief of low back pain, for instance non-specific back pain.

In an embodiment, a “subject” according to the present invention is a mammal. In an embodiment, the subject is a human; in an embodiment, the subject is a male human; in an embodiment, the subject is a female human. In an embodiment, a subject is a dog, a cat, a horse, a primate, or other mammal that may experience back pain and/or needs relief from back pain; in an embodiment, said back pain is low back pain including non-specific low back pain. In an embodiment, a subject of this invention does not have osteoarthritis; in an embodiment, a subject does not have osteoarthritis of the back. In an embodiment, a subject of this invention does not have a diagnosis of osteoarthritis; in an embodiment, a subject does not have a diagnosis of osteoarthritis of the back. In an embodiment, a subject is not overweight (e.g. BMI less than 25, less than 24, less than 23, less than 22, less than 21, less than 20, where BMI is calculated as kg/(height (m))²). In an embodiment, a subject is 1-105 years old, including for instance adult subjects (e.g. 18-90 years old, 25-70 years old). In an embodiment, a subject has a BMI of about 25-29; in an embodiment, a subject has a BMI of about 30-40, for instance 31-40, 32-36, and the like).

In an embodiment, a “composition” of the present invention comprises Terminalia chebula (“Terminalia chebula composition”). In an embodiment, a composition of this invention may comprise, consist essentially of, or consist of, Terminalia chebula, including in particular composition embodiments described throughout this application.

In an embodiment, a composition of this invention is a powder or an extract of T. chebula fruit; in an embodiment, an extract of T. chebula fruit pulp. In the present invention, in an embodiment, an “extract” is an aqueous, alcoholic, or hydro-alcoholic extract of Terminalia chebula such as Terminalia chebula fruit. In an embodiment, an extract is prepared from T. chebula fruit (e.g. pulp) by removing the seeds and treating the fruit pulp with water or an aqueous solution such as phosphate buffered saline (PBS) or other aqueous solution with, for instance, a salt, pH, and/or other chemical component(s) to form the aqueous extract, and then in an embodiment standardized to minimum or maximum amount or a specific range of bioactives of T. chebula fruit, such as discussed in Example 1 below, so as to render the extract consistent at least with regard to those components from one batch to the next. In an embodiment, a T. chebula composition of this invention, such as an extract of Terminalia chebula fruit or powdered Terminalia chebula fruit, comprises, consists essentially of, or consists of chebulinic acid (for instance 15% w/w or more), chebulagic acid (for instance 10% w/w or more), gallic acid (for instance 10% w/w or less), ellagic acid (for instance 10% w/w or less), and other Low Molecular weight Hydrolyzable Tannins (LMwHTs; for instance 12% w/w or more); in an embodiment, with the above measured via HPLC/HPLC-PDA and calculated against an external standard of Chebulinic acid or, for Gallic acid, an external standard of Gallic acid. In an embodiment, an extract of this invention is AyuFlex® as described in Example 1. In an embodiment, an extract is in a solid form, such as a powder, or a liquid or other form as desired. In an embodiment, an extract, for instance a standardized aqueous extract, of T. chebula (e.g. fruit or fruit pulp), comprises low molecular weight hydrolysable tannins (LMwHTs), including chebulinic acid and chebulagic acid combined, not less than 37% w/w on “as is” basis (not on anhydrous basis) with other unidentified LMwHTs not less than 12% w/w on “as is” basis, when analyzed by the analytical method described in Example 1. In addition, in an embodiment, ellagic acid and gallic acid are also present in the extract, for which the analytical results are only reported without any specification, but specifications for these bioactives may also be developed. A different analytical method using reference standards of the bioactives, available from commercial sources, may enable one to identify and quantify other bioactives in the extract. In an embodiment, a composition of this invention comprises at least 25% (w/w) Chebulinic acid+Chebulagic acid as measured by HPLC. In an embodiment, a composition of this invention is a dried Terminalia chebula fruit powder. In an embodiment, a composition of this invention is a dried and standardized Terminalia chebula fruit powder. In an embodiment, a composition of this invention is a blend of dried powders of the fruits of T. chebula, Phyllanthus emblica, and Terminalia bellerica; in an embodiment, the blend is at least 50% w/w T. chebula powder; in an embodiment, the blend is a 1:1:1 blend of T. chebula, Phyllanthus emblica, and Terminalia bellerica powder, where the powder is of T. chebula fruit and/or an extract of T. chebula fruit.

Chemical constituents isolated from T. chebula may vary considerably in type and/or concentration due to a number of factors, e.g., ecological variation, soil variation, and nutrient variation, as well as variations in the process of extraction. In an embodiment, a standardized, potent and therapeutically effective extract of T. chebula is provided in a pharmaceutical or nutraceutical composition having improved properties for the treatment of back pain such as low back pain.

A composition of the present invention may be formulated into nutraceutical or pharmaceutical dosage forms comprising for instance tablets, capsules, powders, liquids, chews, gummies, transdermals, injectables, dietary supplements, topical creams, lozenges, pills, sachets, and so forth. A composition of the present invention may be formulated as a dietary supplement. A composition of the present invention may further comprise one or more excipients, additives, and/or other substances, including for instance microcrystalline cellulose, croscarmellose sodium, magnesium stearate, and/or silicon dioxide.

In an embodiment, a composition of the present invention is AyuFlex®, a standardized extract of Terminalia chebula fruit. In an embodiment, AyuFlex® is certified organic. In an embodiment, AyuFlex® is a dietary supplement. In an embodiment, AyuFlex® is in powder form, having light yellow color and an astringent taste; is at least 37.0% (w/w) LMwHTs (Low Molecular weight Hydrolysable Tannins) as measured by HPLC against a Chebulinic acid standard, including at least 25.0% (w/w) Chebulinic acid+Chebulagic acid (for instance, 15% w/w or more Chebulinic acid and 10% w/w or more Chebulagic acid), and including at least 12% (w/w) other LMwHTs; optionally has Gallic acid (not more than 10%, i.e. 0-10% w/w) and Ellagic acid (not more than 10%, i.e. 0-10% w/w); has a water extractive value of at least 80% (w/w) by gravimetry, has a moisture content of not more than 6.0% (w/w) by Karl-Fischer analysis; has heavy metals (ICP-MS) lead (Pb), arsenic (As), mercury (Hg), and cadmium (Cd) not more than 2 ppm, 2 ppm, 1 ppm, and 1 ppm, respectively; and a microbiological profile of not more than 5000 CFU/g of aerobic bacteria (non-pathogenic), not more than 1000 CFU/g yeast and mold, Salmonella species absent in 10 g, and Escherichia coli, Staphylococcus aureus, Pseudomonas aeruginosa, and Candida albicans each independently absent in 1 g of AyuFlex® powder. In an embodiment, AyuFlex® is stored in original sealed containers at 15-25° C., avoiding light.

In an embodiment, AyuFlex® is in powder form, having light yellow color and an astringent taste; is about 37-80% (w/w) LMwHTs (Low Molecular weight Hydrolysable Tannins) as measured by HPLC, for instance about 40-75%, about 50-70%, about 65-70%, for instance about 67%, including about 40-50% (w/w) Chebulinic acid+Chebulagic acid, for instance about 46.5%, and about 15-25% (w/w) other LMwHTs, for instance about 20.6%; has about 9% Gallic acid and about 8% Ellagic acid as measured by HPLC; has a water extractive value of about 93-94% (w/w) by gravimetry, has a moisture content of about 5% (w/w) by Karl-Fischer analysis; has heavy metals (ICP-MS) lead (Pb), arsenic (As), mercury (Hg), and cadmium (Cd) not more than 0.1 ppm, 0.12 ppm, less than 0.001 ppm, and 0.0.007 ppm, respectively; and a microbiological profile of about 1000 CFU/g of aerobic bacteria (non-pathogenic) (USP 2021), less than about 50 CFU/g yeast and mold (USP 2021), and no detectable Salmonella species in 10 g AyuFlex® powder (USP 2022) or Escherichia coli (USP 2022), Staphylococcus aureus (USP 2021), Pseudomonas aeruginosa (USP 62), or Candida albicans (Custom protocol) in 1 g of AyuFlex® powder. In an embodiment, the AyuFlex® powder is encapsulated, for instance in an amount of 500 mg powder, for oral administration, for instance as used in the below Examples.

Method of Manufacturing of T. chebula Fruit Powder, or T. chebula Extract:

Dried fruit powder: Seeds are removed from the dried fruits, the pulp is dried and milled to a fine powder and the powder is standardized to specifications developed by using the analytical method described in Example 1. Drying techniques may include oven drying, freeze-drying, microwave drying and the like and the milling technique may be hammer milling, micronization and the like.

Fruit extract (liquid or powdered): In an embodiment, a method of manufacturing of a T. chebula fruit extract is described in U.S. Pat. No. 10,500,240, which is incorporated by reference herein to describe a method of manufacturing T. chebula fruit extract to the extent allowed by law. In an embodiment, the extraction process of the current invention includes the steps of providing dried fruits of T. chebula, de-seeding the fruits, pulverizing or grinding the pulp to a powder, extracting the pulp powder with an extraction solvent or solvent mixture, optionally, with heating, to provide a T. chebula enriched liquid extract, optionally concentrating the liquid extract and drying the concentrated liquid extract to provide a hydrolyzable tannoid enriched T. chebula extract powder. In an embodiment, aqueous solvent is preferred. In an embodiment, a particularly preferred solvent is water. Useful extraction temperatures can range from about 25° C. (ambient) to about 90° C. Particularly useful extraction temperatures can range from about 25° C. to about 80° C.

In an embodiment, AyuFlex® is prepared in keeping with the methods described above. In an embodiment, useful extraction times for preparing an extract according to this invention in conjunction with maintaining useful temperatures can range from about 2 hours to about 16 hours. A particularly useful extraction time range at about 25°±5° C. is from about 12 hours to about 16 hours, and at a temperature of 40°±5° C. is from about 2 hours to about 6 hours. Length and temperature of extraction may be varied at atmospheric pressure (i.e., approx. 1 atm). It is contemplated that pressure can be varied in the extraction process, for example, by use of a commercial pressure reactor apparatus.

The extraction process can also include drying the liquid extract to a powder form. Suitable drying methods include spray drying, lyophilization, freeze drying, vacuum drying (with or without heating), evaporation (with or without heating), and concentration under vacuum. Once isolated or obtained, the hydrolyzable tannoid enriched T. chebula extract powder may be processed by any suitable means, including grinding, milling, sieving, sizing, blending and the like. The obtained hydrolyzable tannoid enriched T. chebula extract powder may be prepared in any suitable particle size or particle size range.

Process additives such as microcrystalline cellulose, starch, maltodextrin and the like as carrier materials, anti-adherents such as silicone dioxide, rice bran powder and the like, and preservatives such as sodium benzoate, methyl paraben, propyl paraben, natural preservatives and the like may be added during the extraction process or during the final blending of the dried extract powder.

In an embodiment, ethanol or methanol or a hydro-alcoholic mixture may be used as the solvent system for extraction, as described throughout this application. In an embodiment, a hydro-alcoholic solvent system of this invention contains 1:9 to 9:1 (w/w) ratio of alcohol to water, in an embodiment, the ratio being 3:7 to 7:3 (w/w) alcohol to water. In an embodiment, the alcohol is ethyl alcohol (ethanol) or methyl alcohol (methanol). In an embodiment, the temperature and/or time of extraction is the same for an alcoholic or hydroalcoholic solvent as for an aqueous extraction such as with water, as described above. In an embodiment, alcoholic or hydroalcoholic solvent for extraction of the fruits of T. chebula may yield a different bioactive composition with different active components or different amounts according to the present invention.

“Administering”, “administration”, and the like, according to the present invention refers to providing an effective amount of a Terminalia chebula composition of the present invention to a subject so that the Terminalia chebula chemical constituents/components may reach the subject's bloodstream and/or tissues and/or cells, including for instance tissues and cells of the back and/or related to back pain such as low back pain, and act on the tissues and cells to relieve back pain such as low back pain. In an embodiment, such constituents/components are one or more active components of a Terminalia chebula composition of this invention, such as a standardized composition, with active components as described in Example 1. Administration may be by the subject or by another. Administration may be oral, for instance in the form of a dietary supplement or a pharmaceutical formulation, for instance in the form of a liquid, in the form of a powder (e.g. loose or sachet), and/or in a solid dosage form, in an embodiment in a discrete dose unit, such as a capsule including for instance the Ayuflex® capsules described in Examples 2-5. Administration may also be through parenteral, intramuscular, transdermal, and other physiologically acceptable routes.

“Co-administration” refers to an embodiment of this invention including administering a composition of the present invention with another substance, including for instance, a drug that relieves or treats back pain, or another substance as desired. Examples of drugs that may be co-administered with a composition of the present invention are provided throughout the application. In an embodiment, the present invention does not include the co-administration of any other substance to achieve relief of back pain.

In the present disclosure, an “effective amount” of Terminalia chebula or a Terminalia chebula composition refers to an amount of Terminalia chebula or T. chebula composition of this invention needed to reach a subject's bloodstream and/or bodily tissues and relieve back pain such as low back pain in the subject, as described above. In an embodiment, an effective amount of a standardized Terminalia chebula composition such as AyuFlex® for relieving back pain is 50-4000 mg/day, for instance 50, 100, 200, 300, 400, 500, 600, 700, 800, 900, 1000, 1100, 1200, 1300, 1400, 1500, 1600, 1700, 1800, 1900, 2000, 2500, 3000, 3500, or 4000 mg/day, or any amount or range within said range. In an embodiment, an effective daily amount of AyuFlex® for treating and/or relieving back pain such as low back pain according to this invention in an adult human subject is at least 500 mg, at least 1000 mg, at least 1250 mg, at least 1500 mg, at least 2000 mg, and/or more than 2000 mg AyuFlex® per day; in an embodiment with said amounts capped for instance at 4000 mg daily or another amount indicated as a safe maximum daily dosage, for instance by an applicable regulatory agency or other findings. In an embodiment, an “effective amount” of a standardized Terminalia chebula composition such as AyuFlex® for relieving back pain is about 5 mg/kg body weight of a subject to about 100 mg/kg body weight of a subject; in an embodiment, about 6 to about 50 mg/kg body weight of a subject; in an embodiment, about 6.8 mg/kg body weight of a subject to about 37 mg/kg body weight of a subject.

In an embodiment, the above amounts are effective amounts of this invention for standardized aqueous, alcoholic, or hydro-alcoholic extracts of this invention. In an embodiment, the daily dose range for a powder of dried T. chebula fruits is about 500 mg/day to about 10,000 mg/day, or about 10 mg/kg body weight of a subject to about 200 mg/kg body weight of a subject.

A “dietary supplement” according to the present invention refers to a Terminalia chebula composition of the present invention which is administered as an addition to a subject's diet, which is not a natural or conventional food, which when administered according to the present invention relieves back pain such as low back pain, including for instance non-specific low back pain and/or chronic low back pain. In an embodiment, a dietary supplement comprising an effective amount of Terminalia chebula composition according to the present invention is administered orally. In an embodiment, the dietary supplement is administered daily; in an embodiment, the dietary supplement (or any other composition of the present invention) is administered daily for 1-800 days or more, including any number of days, weeks, or months falling within that range; or for another period of time according to the present invention, including for instance 1 day, 1 week, 2 weeks, 4 weeks, 8 weeks, 12 weeks, more than 12 weeks such as 13 weeks to 5 years, 4 months to 5 years, 6 months to 4 years, 9 months to 4 years, 1-5 years, and so forth. A dietary supplement may be for instance a T. chebula extract or dried T. chebula fruit powder, formulated into various forms, as discussed throughout this application. In an embodiment, AyuFlex® is a dietary supplement according to this invention.

According to the present invention, “relief” from back pain such as low back pain, “relieving” back pain, and the like, refers to reducing and/or eliminating back pain such as low back pain in a subject. In an embodiment, relief from back pain is subjective, with the subject describing a reduction or elimination of back pain such as low back pain. In an embodiment, relief from back pain is from chronic back pain, subacute back pain, or acute back pain. In an embodiment, relief from back pain is as described in Example 2, 3, 4, and/or 5. In an embodiment, relief from back pain is objective, with for instance diagnostic test results such as diagnostic imaging evidencing improvement in back pain during or after administration of a T. chebula composition of this invention, as compared with diagnostic test results from before administration of a T. chebula composition of this invention, or based on other objective measurement(s). In an embodiment, relief from back pain of this invention occurs so long as the subject is administered an effective amount of a T. chebula composition of this invention. In an embodiment, relief from back pain of this invention occurs where the subject experiences a reduction or elimination of back pain such as low back pain during and/or after administration of a T. chebula-composition of this invention. In an embodiment, relief of back pain such as low back pain in a subject refers to resolving the pain, so that the subject no longer experiences the pain, and/or is able to resume activity that previously caused and/or augmented the back pain and/or activity that the back pain had rendered difficult or impossible for the subject to perform.

In an embodiment, relief from back pain according to this invention occurs without administration of any other medicines, including for instance analgesics or anti-inflammatory agents such as acetaminophen (Tylenol®) or ibuprofen (Advil®) or steroids or the like. Accordingly, administration according to this invention may comprise a Terminalia chebula composition (allowing for co-administration of other medicines), consist essentially of a Terminalia chebula composition, or consist of a Terminalia chebula composition of this invention (restricting relief of back pain to only a T. chebula composition of this invention, such as AyuFlex®).

In an embodiment, relief from back pain or relieving back pain includes, once the back pain is reduced or eliminated, preventing the recurrence of back pain in the subject, in an embodiment with continued administration of a Terminalia chebula composition, or in an embodiment, without continued administration. In an embodiment, relief from back pain or relieving back pain includes providing continued relief from back pain such as low back pain in a subject, in an embodiment with continued administration of a T. chebula composition, or in an embodiment, without continued administration.

In an embodiment, “treatment”, “treating” back pain, and the like, according to this invention includes administering a T. chebula composition of this invention to relieve back pain. Such treatment includes relieving back pain for instance by reducing or eliminating it, as discussed throughout this application. In an embodiment, treatment of back pain includes improving the underlying cause of the back pain, including for instance resolving or appearing to resolve the underlying cause. In an embodiment, treatment and/or relief from back pain according to the present invention is in a subject that does not suffer from osteoarthritis, for instance, a subject that does not have a diagnosis of osteoarthritis from a medical professional, including for instance a subject that does not have a diagnosis of osteoarthritis in the subject's back. In an embodiment, references to treatment or relief of back pain are or may be interchangeable.

### EXAMPLES

The present invention may be further understood in connection with the following Example and embodiments. The following non-limiting Example and embodiments described throughout this application are provided to illustrate the invention.

### Example 1

### Assays

### Analytical Methodology

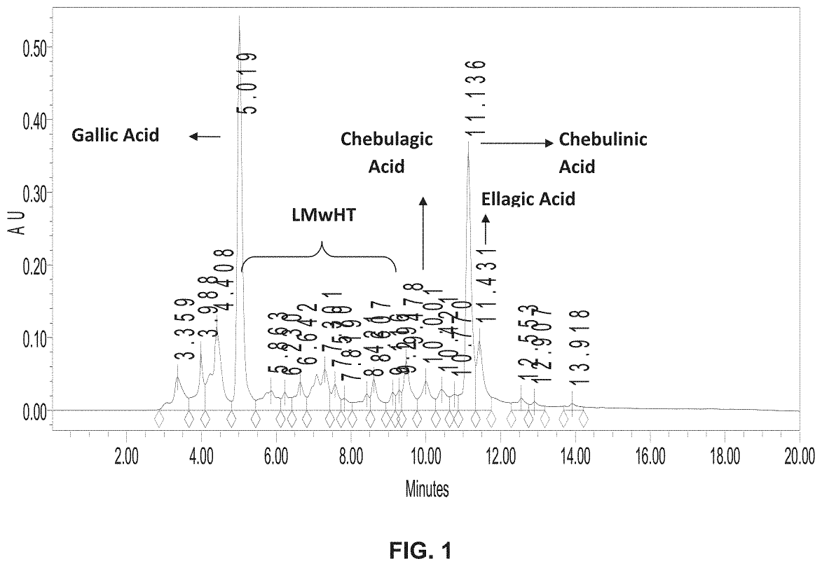

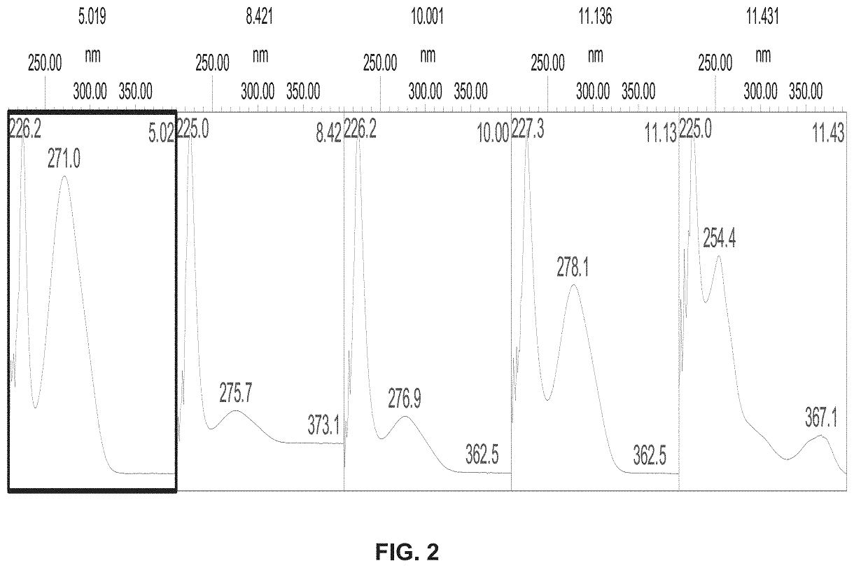

Standardized aqueous extracts of T. chebula fruit (AyuFlex®) were and are analyzed to identify and confirm amounts of active components chebulinic acid, chebulagic acid, other LMwHTs, gallic acid, and ellagic acid. In this Example, chebulinic acid, chebulagic acid, and other LMwHTs in the extract are analyzed by HPLC (High Pressure Liquid Chromatography) and HPLC-PDA and quantified by using the calibration curve of an external chebulinic acid standard. See for instance FIGS. 1 and 2. Gallic acid and Ellagic acid are analyzed by HPLC and quantified by using a calibration curve of, respectively, Gallic acid and Ellagic acid. This Example displays typical analytical methods, results, and expected standards for AyuFlex®.

### Analysis

### Sample Preparation

T. chebula powdered aqueous extract (AyuFlex®) (50 mg) was dissolved in Milli-Q® water (50 ml) by shaking for 5 minutes. The resulting solution had a concentration of 1 mg/ml. It was diluted with Milli-Q® water to obtain a concentration of 0.5 mg/ml and this solution was used for the HPLC analysis. Milli-Q® water refers to water that has been purified using resin filters and deionized to a high degree by a water purification system manufactured by Millipore Corporation (Millipore-Sigma, Burlington, Mass., USA). The system monitors the ion concentration by measuring the electrical resistance of the water. Higher resistance means fewer charge carrying ions.

T. chebula dried fruit powder, about 10 g, may be extracted with purified water at 40° C.-80° C. in a rotovapor using the same procedure as described above for extraction, filtered, and the filtrate dried either in an oven or in a spray dryer to get powdered extract. 50 mg of this extract powder may be dissolved in Milli-Q® water (50 ml) by shaking for 5 minutes. The resulting solution would have a concentration of 1 mg/ml. It may be diluted with Milli-Q® water to obtain a concentration of 0.5 mg/ml and this solution may be used for HPLC analysis. Other T. chebula compositions of this invention may be analyzed according to this Example.

### HPLC Conditions

The column used for analysis is a reverse phase LiChrosorb® RP-18 (Particle size 5 μm, 4×250 mm) column, Merck KGaA; Germany, with a reverse phase guard column.

Column temperature: Ambient

Eluent: [A]: 1% Acetic acid in water, [B]: 1% Acetic acid in Acetonitrile

Flow rate: 0.7 ml/min

Run time: 20 min

Gradient program is mentioned below in Table 1:

TABLE 1
Gradient Program

| Time (min) | Solvent A (% v/v) | Solvent B (% v/v) |
|---|---|---|
| 0 | 90 | 10 |
| 15 | 50 | 50 |
| 16 | 90 | 10 |
| 20 | 90 | 10 |

Detection UV 270 nm
Injection volume 20 μl (with a loop injector)
Equipment Waters HPLC 2695 with PDA Detector (Waters™ 2996, Photodiode Array Detector), evaluation with Empower software
Reagents Pdt. No. UN-1648 Acetonitrile (Merck), Pdt No. 93956 Water for HPLC (Merck)
Evaluation Method with external standard and evaluation of area of peaks using respective calibration equation.

### External Standard

### A. Preparation of Linear Regression Equation for Chebulinic Acid

Reference standard of chebulinic acid (98% w/w pure, isolated from T. chebula fruits using Sephadex G-50 (Amersham Bioscience, formerly of Buckinghamshire, UK) and Low Pressure Chromatography (Bio-Rad, DesPlaines, Ill.)), was dissolved in distilled water to prepare reference standard solutions of five different concentrations (12.5-400 μg/ml), required for the preparation of the calibration curve. These solutions were subjected to HPLC analysis. The peak areas were calculated for each dilution, and the respective concentrations were plotted against the peak area. Chebulinic acid, chebulagic acid, chebulinic acid equivalents and chebulagic acid equivalents were quantified using a regression equation of the calibration curve obtained as follows:

Y=30167x−217853 EQN. I

with a correlation coefficient of 0.999, where Y is the peak area and X is the concentration in μg/ml.

Gallic acid reference standard (Phyproof® Reference Standard, catalogue #PHL89198, CAS No. 149-91-7, (HO)₃C₆H₂CO₂H, MW 170.12, Millipore-Sigma, Burlington, Mass.) was dissolved in methanol to prepare five different concentrations (5-100 μg/ml) required for preparation of a calibration curve. The amount of gallic acid in each solution was determined using a regression equation of the calibration curve:

Y=85092x+25514 EQN. II

with a correlation coefficient of 0.999, where Y is the peak area and X is the concentration in μg/ml.

Ellagic acid reference standard (Phyproof® Reference Standard, catalogue #PHL89653, CAS No. 476-66-4, C₁₄H₆O₈, MW 302.19, Millipore-Sigma, Burlington, Mass.) was dissolved in methanol to prepare five different concentrations (5-100 μg/ml), required for preparation of a calibration curve. The amount of ellagic acid in each solution was determined using a regression equation of the calibration curve:

Y=132665x−439279 EQN. III

with a correlation coefficient of 0.999, where Y is the peak area and X is the concentration in μg/ml.

### Calculations

i) Chebulagic acid: The area of the peak appearing at t_{R} 10.00 minutes was attributed to chebulagic acid and the amount calculated using the above-mentioned calibration equation of chebulinic acid (Y=30167x−217853) and the formula as follows.

$$Percentage of Chebulagic acid = \frac{\begin{matrix}Amount of chebulagic acid \\ obtained using calibration equation ( μg )\end{matrix}}{Amount of sample injected ( μg )} \times 100 \qquad EQN . IV$$

ii) Chebulinic acid: The area of the peak appearing at t_{R} 11.13 minutes was considered as Chebulinic acid and the amount calculated using the above-mentioned calibration equation of Chebulinic acid (Y=30167x−217853) and the formula as follows.

$$Percentage of Chebulagic acid = \frac{\begin{matrix}Amount of chebulagic acid \\ obtained using calibration equation ( μg )\end{matrix}}{Amount of sample injected ( μg )} \times 100 \qquad EQN . V$$

iii) Other LMwHTs: The sum of the area of peaks appearing between t_{R} 5-9 minutes were added and the amount of other LMwHTs calculated using the calibration equation of Chebulinic acid (Y=30167x−217853) and the formula as follows.

$$Percentage of Other LMwHTs = \frac{\begin{matrix}Amount of other LMwHTs \\ obtained using calibration equation ( μg )\end{matrix}}{Amount of sample injected ( μg )} \times 100 \qquad EQN . VI$$

iv) Gallic acid: The area of the peak appearing at t_{R} 5.01 minutes was considered as Gallic acid and the amount calculated using the above-mentioned calibration equation of Gallic acid (Y=85092x+25514) and the formula as follows.

$$Percentage of Gallic acid = \frac{\begin{matrix}Amount of Gallic acid \\ obtained using calibration equation ( μg )\end{matrix}}{Amount of sample injected ( μg )} \times 100 \qquad EQN . VII$$

(v) Ellagic acid: The area of the peak appearing at t_{R} 11.43 minutes is considered as Ellagic acid and the amount calculated using the above-mentioned calibration equation of Ellagic acid (Y=132665x−439279) and the formula as follows.

$$Percentage of Ellagic acid = \frac{\begin{matrix}Amount of Ellagic acid \\ obtained using calibration equation ( μg )\end{matrix}}{Amount of sample injected ( μg )} \times 100 \qquad EQN . VIII$$

FIGS. 1 and 2 show typical a HPLC chromatogram and PDA spectra of the above analysis. Reading FIG. 1 peaks from left to right, FIG. 1 shows peaks at 3.359, 3.988, 4.408, 5.019 (Gallic Acid), 5.863, 6.230, 6.642, 7.301, 7.570, 7.819, 8.421, 8.607, 9.116, 9.291, 9.478, 10.001 (Chebulagic Acid), 10.421, 10.770, 11.136 (Chebulinic Acid), 11.431 (Ellagic Acid), 12.553, 12.907, 13.918, with LMwHTs indicated after 5.019 (Gallic Acid) up to 9.478. FIG. 2 first box from the left (5.019) shows peaks at 226.2 and 271.0; second box from left (8.421) shows peaks at 225.0 and (small peak) 275.7: middle box (10.001) shows peaks at 226.2 and (small peak) 276.9; fourth box from left (11.136) shows peaks at 227.3 and 278.1; and far right box (11.431) shows peaks at 225.0, 254.4, and (small peak) 367.1.

In addition, moisture content of AyuFlex® of ≤6% is determined by a method according to Karl Fischer: Using M/s Popular India's Karl Fischer titrimeter Model 761E and Reagents Cat. No. 38524 Karl Fischer solution pyridine-free titrant and Cat. No. 27985 di-sodium tartarate dehydrate GR (ThermoFisher Scientific Qualigens Chemicals, Maharashtra, INDIA), using potentiometric titration/indication.

Specifically, methanol was taken in the titration vessel. The auto zero burette was filled to zero level and the K.F. titrimeter was switched on. The moisture content in the methanol was removed as or pressing START K.F. reagent was added automatically until complete moisture was removed from the expected dry methanol. The known quantity (e.g. 200 mg) of finely powdered or liquid sample to be measured was taken in. Burette reading was taken before pressing the START push button switch or burette may be filled to ml position with K.F. Burette reading was taken and START switch was pressed. K.F. reagent started flowing in the titration vessel (RUN and ADD LEDs glowed) and continued until the END point was reached. At the END point the instrument waited for 25-30 seconds. If no moisture was detected within that period the END lamp (Red) would glow and the titration was complete. The final burette reading was taken and the volume of K.F. reagent used (e.g. 5 ml) identified. The % moisture content of the sample was calculated as follows:

$$\% Moisture Content = \frac{K . F . ( ml ) \times 100}{Weight of the sample ( mg )} \qquad EQN . IX$$

where MDF=Moisture determining factor of K.F. (normal 5 mg/ml), KF (ml)=K.F. reagent used in milliliters. If liquid samples were added then weight of the sample (mg)=Vol. of the sample (ml)×specific gravity of sample. If necessary to determine the MDF (moisture-determining factor) of the K.F. reagent, MDF of K.F. was calculated by a sodium tartarate dihydrate method: Dry methanol (25 ml) was taken in titration vessel and titrated with K.F. reagent (Cat. No. 35824). The pure Sodium tartarate dihydrate (0.2 g) (15.66% water) was accurately weighed. It was stirred and titrated again with the K.F. reagent. The salt dissolved completely before titration was completed.
To calculate the mg of water or water equivalent to 1 ml of the K.F. reagent from the formula:

$$Milligram of water per ml of K . F . = \frac{mg of sample \times 0.1566}{mg of reagent K . F .} \qquad EQN . X$$

Water-soluble extracted value ≥90.0% was determined as follows: Sample (1 g) was accurately weighed and dissolved in 100 ml distilled water in a flask. The solution was sonicated for 10 minutes and warmed for 5 minutes on a steam bath (80° C.+5° C.). After cooling, the solution was centrifuged at 8000 rpm for 12 minutes. The supernatant was separated and evaporated (10 ml) on water bath on a pre-weighed petri dish. After evaporation, the petri dish was placed in a vacuum for 1 hour for ensuring complete evaporation of water. The weight was taken and from the weight difference, water soluble extractive value of the sample was calculated.

### Results and Discussion

HPLC assays and HPLC-PDA spectra confirm the identity and amount of active component standards in AyuFlex® as follows: Total Low Mwt Hydrolyzable Tannins (≥37.0% w/w), including i) chebulinic acid (≥15.0% w/w), ii) chebulagic acid (≥10.0% w/w), iii) Other LMwHTs (>12.0% w/w), iv) Gallic acid (≤10.0% w/w), and v) Ellagic acid (≤10.0% w/w). In addition, water-soluble extractive value was ≥80% w/w and Moisture content (Karl Fischer) ≤6.0% w/w. Also, Heavy Metal analyses (ICP-MS USP <730>) of AyuFlex® show not more than (≤)2 ppm Lead (Pb), ≤2 ppm Arsenic (As), ≤1 ppm Mercury (Hg), and ≤1 ppm Cadmium (Cd), and Microbiological Tests show Aerobic bacteria (non-pathogenic) ≤5000 CFU/g <USP2021>, Yeast and mold ≤1000 CFU/g <USP2021>, Escherichia coli absent in 1 g <USP2022>, Staphylococcus aureus absent in 1 g <USP2021>, Pseudomonas aeruginosa absent in 1 g <USP62>, Salmonella species absent in 10 g <USP2022>, and Candida albicans absent in 1 g of AyuFlex® extract of this invention.

The above and other descriptions of embodiments of this invention throughout this application is not intended as limiting. For instance, without being bound by theory, it is conceivable that a different method of analysis, using different bioactive reference standards, may yield different bioactive composition and/or different amounts of the bioactives in the products of the present invention. Also, in an embodiment, an analytical method may be modified as more sophisticated analytical methods and more diverse bioactive reference standards become available. The description of other embodiments of this invention, as discussed throughout this application, are also not intended as limiting.

### Examples 2-5

Several human subjects suffering from back pain were administered an effective amount of a T. chebula composition according to the present invention (AyuFlex®, an aqueous extract of T. chebula fruit in powdered and standardized form, orally administered in a capsule containing 500 mg of the extract). Each subject reported that the T. chebula composition relieved back pain, as discussed in Examples 2-5. Subject weights ranged from 54.5 kg to 72.7 kg, and daily doses ranged from 500 mg to 2000 mg per day of AyuFlex®, which may be calculated at a dose range of about 6.88 mg AyuFlex®/kg body weight of a subject to about 36.7 mg AyuFlex®/kg body weight of a subject. Each of the below Examples shows successful treatment of back pain with T. chebula compositions of this invention, with such treatment resulting in significant relief from back pain, for instance by reducing or eliminating back pain.

### Example 2

A subject according to this invention, an adult man, 27 years of age and weighing about 145 pounds, reported that AyuFlex® administration relieved his low back pain.

Prior to administration of AyuFlex®, the subject reported a history of suffering from chronic low back pain, described by the subject as sciatic pain, as well as from patellar tendonitis, for the past ten years. The subject indicated his belief that the low back pain was due to a very active lifestyle that included lifting heavy weights. He previously attempted to relieve the chronic low back pain with numerous rounds of physical therapy and rehab-based regimens, but was only provided with temporary relief until he resumed exercise.

The subject began oral administration of 2 (two) 500 mg AyuFlex® capsules daily to treat his low back pain. The subject continued the daily administration of AyuFlex® for 2-3 months, and reported his low back and knees were far less inflamed and painful than before he began administration of AyuFlex®. He continued the daily administration of AyuFlex® for several more months, and after about 1 year total of AyuFlex® administration, reported he was able to resume his weight-lifting activities, and felt as if he never had any sciatic issues to begin with. The subject reported he did not take any other pain-relieving medications while taking AyuFlex®, and is continuing to take AyuFlex® daily.

### Example 3

A subject according to this invention, an adult woman, 35 years of age and weighing 160 pounds, reported that AyuFlex® administration relieved her low back pain.

Prior to administration of AyuFlex®, the subject reported a history of back pain described by the subject as sciatic pain, beginning when she was young, around ten years old. She was a gymnast for many years, and as a result injured her back. She was diagnosed with small compression fractures and disk bulges in her lower back. Throughout her teen years, the subject underwent intermittent physical therapy, and also sought treatment from a chiropractor for two years. Although the subject reported some temporary relief from these treatments, her pain was not resolved.

The subject further reported that, due to lack of exercise over the years, her sciatic pain became extremely severe. She described the pain as starting at her lower back and continuing down her leg, her calf, and into her foot. Within a month of the onset of these severe symptoms, the subject described having only one or two days where she was pain-free. She reported relying heavily on acetaminophen (Tylenol®) and ibuprofen (Advil®) to ease her pain, as she was often not able to sleep due to the sharp sciatic pain. The subject reported these medicines only relieved her pain for a few hours before the pain resurfaced. Along with the sciatic pain, for a few months before beginning administration of Ayuflex®, the subject reported experiencing stiffness in her lower back as well. She reported needing to bend her back to walk for a few minutes after she would stand.

The subject began oral administration of Ayuflex®, taking a 500 mg capsule twice daily for about 2 months, and then recently chose to increase the dosage to 1000 mg (2 capsules) twice daily. The subject describes a vast reduction of low back pain with AyuFlex® administration, stating that the majority of her sciatic pain and the stiffness in her back has subsided. The subject reported sometimes she still experiences tenderness in her legs and calf, but the pain is now well managed (relieved), and she no longer needs to take acetaminophen (Tylenol®) or ibuprofen (Advil®) for pain management.

### Example 4

A subject according to this invention, an adult woman, 33 years of age and weighing 120 pounds, reported that AyuFlex® administration relieved her lower back pain.

Prior to administration of AyuFlex®, the subject reported a history of back pain, beginning in 2017. The subject reported using ibuprofen for instant relief.

Describing the pain as “too bad” and “unbearable”, the subject began oral administration of AyuFlex® capsules. For the first three days, the subject took one 500 mg AyuFlex® capsule twice daily (1000 mg daily), while also taking one cyclobenzaprine (Flexeril®) 5 mg tablet and one Sensoril® (Withania somnifera, ashwagandha extract) 250 mg capsule at bed time, and reported that the AyuFlex® started giving her relief from the pain. Thereafter, the subject stopped taking cyclobenzaprine (Flexeril®) 5 mg tablet but continued to take 1 (one) 500 mg AyuFlex® capsule and one Sensoril® 250 mg capsule daily. The subject noted that she also had continuous postpartum foot pain and that AyuFlex® administration proved to be a “miracle” medicine, providing relief of this pain too.

The subject also reported taking one 250 mg Sensoril® (Withania somnifera, Ashwagandha extract) for sleep issues a week before administration of AyuFlex®, but reported the Ashwagandha extract provided no benefit with her back pain, and that the pain was still the same.

### Example 5

A subject according to this invention, an adult woman, 63 years of age and weighing 130 pounds, reported that AyuFlex® administration relieved her low back pain.

Prior to administration of AyuFlex®, the subject reported a history of back pain, described by the subject as sciatic pain, starting during her teenage years. Once she started working at a post office, the subject reported, her lower back and sciatic pain worsened due to heavy lifting. The subject reported that the sciatic pain would start in her lower back and continued down the leg and calf. The subject reported that she was diagnosed with disk bulges in her neck and lower back. The subject underwent physical therapy intermittently, which provided some temporary relief, but did not resolve the pain. To find relief, the subject took acetaminophen (Tylenol®) or ibuprofen (Advil®); however, the subject reported that these medicines also only relieved the pain for a few hours. Also, the subject reported a diagnosis of Arthritis and Osteoporosis around ten years ago. Due to this, the subject reported, her fingers would swell, and she had knee pain as well.

The subject began oral administration of Ayuflex®, taking a 500 mg capsule twice daily for about the last 2 years and, during that time, she also took acetaminophen (Tylenol®) or ibuprofen (Advil®) tablets. The subject reported that her lower back and sciatic pain have decreased tremendously and she no longer has to rely on acetaminophen (Tylenol®) or ibuprofen (Advil®) to relieve her pain, and is continuing to take one 500 mg AyuFlex® capsule twice daily.

The subject also reported that the swelling of her fingers and her knee pain remained, but did not worsen over time.

### Discussion

T. chebula compositions have not been previously studied with regard to their effects in relieving back pain, either acute or chronic. The initial thought was that efficacy of the T. chebula extract in relieving low back pain is due to its anti-osteoarthritic efficacy, demonstrated in earlier studies. However, one of the subjects discussed in the Examples was taking another product—Sensoril®, an aqueous extract of the roots plus leaves of Withania somnifera (ashwagandha) plant, which has been shown to be nearly twice as effective in relieving knee osteoarthritis as the T. chebula extract of the present invention and shown to decrease pain threshold force and pain threshold time by a hot air pain model and mechanical pain model. Sensoril® capsules, at 125 mg and 250 mg twice daily dosage, have been shown to be effective in treating osteoarthritis, and at 250 mg twice daily dosage, being nearly as effective as AyuFlex® at 500 mg twice daily dosage. As Sensoril® did not show any benefit for relieving low back pain in this instance, a product that may work well in treating osteoarthritis may not work for relieving back pain. G. S. H. RAMAKANTH et al., Integr. Med. 7(3):151-157 (JulSep 2016); NALINI et al., Res. J. Life Sci. 01(02):1-6 (May 2013); NOOKALA et al., J. Clin. Diagn. Res. 13(1): FC01-FC04 (January 2019). The Withania somnifera extract did not relieve low back pain in the subject, while the T. chebula extract of the present invention did. Without being bound by theory, it appears that a product which works in relieving knee osteoarthritis and pain caused by external forces may not necessarily relieve low back pain. The treatment of back pain and relief of back pain by administration of a T. chebula composition according to the present invention provided significant relief from back pain by significant reduction or elimination of back pain in subjects after administration of the T. chebula composition.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%: in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All method steps described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification the present invention has been described in relation to certain embodiments thereof, and many details have been put forth for the purposes of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

1. A method of relieving back pain in a subject in need thereof, comprising the steps of:
a. providing a composition comprising Terminalia chebula fruit to a subject in need of back pain relief, and
b. administering the composition to the subject to relieve back pain.

2. The method of claim 1, wherein in said administering step, an effective amount of the composition is administered to the subject to deliver the Terminalia chebula fruit composition and/or its chemical constituents to the subject's bloodstream and bodily tissues to act on the subject's back and related tissues and relieve the subject's back pain.

3. The method of claim 1, wherein the composition is an aqueous, alcoholic, or hydroalcoholic extract of Terminalia chebula fruit, in a standardized form and in a dried powdered form or a liquid form.

4. The method of claim 1, wherein the back pain is low back pain.

5. The method of claim 1, wherein the composition is a dietary supplement or a pharmaceutical formulation.

6. The method of claim 1, wherein said back pain is chronic low back pain.

7. The method of claim 1, wherein said back pain is sciatic, referred, non-specific, and/or severe low back pain.

8. The method of claim 1, wherein said Terminalia chebula fruit composition is administered for at least 4 months to relieve low back pain.

9. The method of claim 1, wherein said Terminalia chebula fruit composition is administered in a daily amount of 50-4000 mg/day.

10. The method of claim 9, wherein said daily amount is 500, 1000, 1500, or 2000 mg/day.

11. A method of treating back pain in a subject in need thereof, comprising the steps of:
a. providing a composition comprising Terminalia chebula fruit to a subject in need of back pain relief, and
b. administering the composition to the subject to relieve back pain.

12. The method of claim 11, wherein in said administering step, an effective amount of the composition is administered to the subject to deliver the Terminalia chebula fruit composition or components thereof to the subject's bloodstream and bodily tissues to act on the subject's back and related tissues and relieve the subject's back pain.

13. The method of claim 12, wherein the composition is an aqueous, alcoholic, or hydroalcoholic extract of Terminalia chebula fruit, in a standardized form and in a dried powdered form or a liquid form.

14. The method of claim 11, wherein the back pain is low back pain.

15. The method of claim 11, wherein the composition is a dietary supplement or a pharmaceutical formulation.

16. The method of claim 11, wherein said back pain is chronic low back pain.

17. The method of claim 11, wherein said back pain is sciatic, referred, non-specific, and/or severe low back pain.

18. The method of claim 11, wherein said Terminalia chebula fruit composition is administered for at least 4 months to relieve low back pain.

19. The method of claim 11, wherein said Terminalia chebula fruit composition is administered in a daily amount of 50-4000 mg/day.

20. The method of claim 19, wherein said daily amount is 500, 1000, 1500, or 2000 mg/day.
